(19) United States
(12) Patent Application Publication
(10) Pub. No.: US 2026/0077167 A1
(43) Pub. Date: Mar. 19, 2026

(54) BALLOON FOR BALLOON CATHETER, BALLOON CATHETER INCLUDING SAME, AND METHOD FOR PRODUCING BALLOON CATHETER

(71) Applicant: KANEKA CORPORATION, Osaka-shi, Osaka (JP)

(72) Inventors: Masahiro KOJIMA, Settsu-shi (JP); Takahisa HAMABUCHI, Osaka-shi (JP); Yoshinori NAKANO, Settsu-shi (JP); Masato TSUEDA, Settsu-shi (JP)

(73) Assignee: KANEKA CORPORATION, Osaka-shi, Osaka (JP)

(21) Appl. No.: 18/996,744

(22) PCT Filed: Jul. 7, 2023
(86) PCT No.: PCT/JP2023/025252

§ 371 Date: Jan. 17, 2025

(30) Foreign Application Priority Data
Aug. 2, 2022 (JP) 2022-123511

(51) Int. Cl. A61M 25/10

(52) U.S. Cl. CPC A61M 25/1036; A61M 25/1029; A61M 2025/1086; A61M 2207/00

(57) ABSTRACT

Disclosed is a balloon for a balloon catheter in which the protrusion part provided on the balloon can easily bite into the stenosis, thereby efficiently dilating the stenosis. A balloon (2) for a balloon catheter having a balloon body (20) and a protrusion part (28) that projects outward from the outer surface of the balloon body (20) in the radial direction (y1) and extends in the longitudinal axis direction (x1); the protrusion part (28) having a tip region (28t) including an outer end (28T) and a base region (28b) located inward from the tip region (28t), the balloon body (20) and the protrusion part (28) being composed of the same material, and the surface roughness of the base region (28b) is smaller than that of the tip region (28t) where the surface roughness if measured for a reference length in a direction perpendicular to the longitudinal axis direction (x1).

### TECHNICAL FIELD

The present invention relates to a balloon for a balloon catheter, a balloon catheter including the same, and a method for producing the balloon catheter using a tubular parison made of a resin.

### BACKGROUND ART

Diseases such as angina pectoris and myocardial infarction are caused by the formation of stenotic areas hardened by calcification and other factors in inner walls of blood vessels. One of the treatments for these diseases is angioplasty, in which a balloon catheter is used to dilate the stenotic area. The angioplasty is a minimally invasive therapy that does not require an open chest procedure like bypass surgery and is widely used.

In the angioplasty, conventional balloon catheters may have difficulties in dilating stenotic areas hardened by calcification and other factors. Alternatively, a method is also used to expand the stenotic areas by placing a stent, a device that expands the stenotic areas, but in some cases, for example, after this treatment, a neointima of the blood vessel proliferates excessively and the blood vessel narrows again, causing an ISR (In-Stent-Restenosis) lesions may occur. The neointima in ISR lesions is soft and the surface is slippery, so a standard balloon catheter may cause the balloon to shift out of the lesion site during balloon dilation to damage the vessel.

As balloon catheters that can dilate a stenosis even in such calcified or ISR lesions, balloon catheters with a protrusion, blade or scoring element on the balloon to bite into the stenosis have been developed. For example, Patent document 1 discloses a balloon having a protrusion, and a method for producing a balloon which includes a step of forming a protrusion by welding at least a portion of the adjacent inner surfaces to each other at the part where the inner surfaces of the balloon are arranged facing each other. Patent document 2 discloses a balloon having a gather, which is a protrusion part, and a method of forming a gather on a balloon using a mold.

### RELATED ART DOCUMENT

### Patent Document

Patent document 1: JP 2017-12678 A

Patent document 2: JP 2005-511187 A

### SUMMARY OF THE INVENTION

### Problems to be Solved by the Invention

However, although the above-described protrusion part of conventional balloons comes into contact with the stenotic area, it does not sufficiently penetrate into hardened calcified lesions or slippery ISR lesions, leaving room for improvement in terms of efficiently dilating the stenosis.

In view of the above circumstances, the objective of the present invention is to provide a balloon for a balloon catheter, and a balloon catheter equipped with the same, in which the protrusion part provided on the balloon can easily bite into the stenosis, thereby efficiently dilating the stenosis, as well as a method for producing such a balloon catheter.

### Means for Solving the Problems

A balloon for a balloon catheter in accordance with an embodiment of the present invention that can solve the above problem is as follows.

[1] A balloon for a balloon catheter having a longitudinal axis direction and a radial direction, comprising: a balloon body having an outer surface and an inner surface; a protrusion part that projects outward from the outer surface of the balloon body in the radial direction and extends in the longitudinal axis direction, wherein the protrusion part comprises a tip region including an outer end in the radial direction and a base region located inward from the tip region in the radial direction in a cross-section perpendicular to the longitudinal axis direction; the balloon body and the protrusion part are composed of the same material; and a surface roughness of the base region is smaller than a surface roughness of the tip region where a surface roughness of the protrusion part is measured for a reference length in a direction perpendicular to the longitudinal axis direction.

The balloon for a balloon catheter in accordance with embodiments of the present invention is preferably the following [2] or [3].

[2] The balloon for a balloon catheter according to [1], wherein a surface roughness of the balloon body is larger than the surface roughness of the base region where the surface roughness of the protrusion part and the surface roughness of the balloon body are measured for a reference length in a direction perpendicular to the longitudinal axis direction.

[3] The balloon for a balloon catheter according to [1], wherein a surface roughness of the balloon body is larger than the surface roughness of the tip region where the surface roughness of the protrusion part and the surface roughness of the balloon body are measured for a reference length in a direction perpendicular to the longitudinal axis direction.

The present invention also provides the following.

[4] A balloon catheter comprising the balloon for a balloon catheter according to any one of the above [1] to [3].

The present invention further provides a first method for producing the balloon catheter according to the above [4]. The first method for producing the balloon catheter in accordance with an embodiment of the present invention is as follows.

[5] A method for producing the balloon catheter according to [4], comprising: preparing a parison having a tubular shape and composed of resin; preparing a mold having a longitudinal axis direction and a radial direction, and having an inner cavity extending in the longitudinal axis direction through which the parison is to be inserted, the inner cavity consisting of a groove part recessed outwardly in the radial direction and extending in the longitudinal axis direction and a cylindrical wall part other than the groove part; inserting a cathode conductive member having an outer shape along the cylindrical wall part in a cross-section in the longitudinal axis direction into the inner cavity of the mold to bring the cathode conductive member into contact with the cylindrical wall part without contacting the groove part; connecting the cathode conductive member to a cathode of a power source and connecting an outer wall of the mold to an anode of the power source; introducing an electrolytic solution into the inner cavity of the mold, outside the cathode conductive member in the radial direction; supplying power from the power source to perform electrochemical polishing, removing the electrolytic solution and removing the cathode conductive member from the inner cavity of the mold, followed by inserting the parison into the inner cavity of the mold; and introducing a fluid into a lumen of the parison to inflate the parison and bringing an outer wall of the parison into contact with the groove part and the cylindrical wall part of the mold, wherein in a cross-section perpendicular to the longitudinal axis direction, the groove part has a groove tip region with a longer distance from a figure center of the inner cavity of the mold in the radial direction and a groove base region with a shorter distance from the figure center in the radial direction than that of the groove tip region.

The first producing method in accordance with embodiments of the present invention is preferably the following [6].

[6] The method according to [5], wherein no gap is formed between the cathode conductive member and the cylindrical wall part when the cathode conductive member is brought into contact with the cylindrical wall part.

The present invention further provides a second method for producing the balloon catheter according to the above [4]. The second method for producing the balloon catheter in accordance with an embodiment of the present invention is as follows.

[7] A method for producing the balloon catheter according to [4], comprising: preparing a parison having a tubular shape and composed of resin; preparing a mold having a longitudinal axis direction and a radial direction, and having an inner cavity extending in the longitudinal axis direction through which the parison is to be inserted, the inner cavity consisting of a groove part recessed outwardly in the radial direction and extending in the longitudinal axis direction and a cylindrical wall part other than the groove part; inserting the parison into the inner cavity of the mold; heating the mold; and introducing a fluid into a lumen of the parison to inflate the parison and bringing an outer wall of the parison into contact with the groove part and the cylindrical wall part of the mold, wherein in a cross-section perpendicular to the longitudinal axis direction, the groove part has a groove tip region with a longer distance from a figure center of the inner cavity of the mold in the radial direction and a groove base region with a shorter distance from the figure center in the radial direction than that of the groove tip region; in a cross-section perpendicular to the longitudinal axis direction, the mold has a first region with a low thermal conductivity, a second region with a higher thermal conductivity than the first region, and a third region with a higher thermal conductivity than the second region; and in a cross-section perpendicular to the longitudinal axis direction, the first region is located at the cylindrical wall part, the second region is located at the groove tip region, and the third region is located at the groove base region.

The second producing method in accordance with embodiments of the present invention is preferably the following [8] or [9].

The method according to [7], wherein the first region is composed of a first member formed from a material (L) with a low thermal conductivity, the second region and the third region include a second member formed from a material (H) with a higher thermal conductivity than the material (L), and a thickness of the second material of the third region in the radial direction is greater than a thickness of the second material of the second region in the radial direction.

[9] The method according to [8], wherein the average thickness of the second member of the third region in the radial direction is 1.2 times or greater the average thickness of the second member of the second region in the radial direction.

### Effects of the Invention

According to the above-described balloon for a balloon catheter and the method for producing the balloon catheter, the entire protrusion part can easily bite into a stenosis formed in a lesion, such as calcified hardened lesion, enabling efficient incision of the stenosis. Furthermore, even in slippery lesion areas, the protrusion part can be inserted without shifting from the intended position, making it easier to incise the intended area.

### BRIEF DESCRIPTION OF THE DRAWINGS

FIG. 1 is a side view of a balloon catheter in accordance with one embodiment of the present invention.

FIG. 2 is a perspective view of a balloon for a balloon catheter in accordance with one embodiment of the present invention.

FIG. 3 is a cross-sectional view perpendicular to the longitudinal axis direction of a balloon for a balloon catheter in accordance with one embodiment of the present invention.

FIG. 4 is a cross-sectional view showing a variation of FIG. 3.

FIG. 5 is an example of an image obtained by a measurement of surface roughness of a protrusion part of a balloon for a balloon catheter in accordance with one embodiment of the present invention.

FIG. 6 is a perspective view of a parison in accordance with one embodiment of the present invention before inflation.

FIG. 7 is a cross-sectional view in the longitudinal axis direction of a first mold used in a first producing method in accordance with one embodiment of the present invention.

FIG. 8 is a VIII-VIII cross-sectional view of the first mold shown in FIG. 7.

FIG. 9 is a cross-sectional view perpendicular to the longitudinal axis direction when a cathode conductive member is inserted into an inner cavity of the first mold shown in FIG. 8.

FIG. 10 is a schematic view explaining the first method in accordance with one embodiment of the present invention.

FIG. 11 is a XI-XI cross-sectional view of FIG. 10.

FIG. 12 is a cross-sectional view perpendicular to the longitudinal axis direction of a second mold used in a second producing method in accordance with one embodiment of the present invention.

FIG. 13 is a cross-sectional view showing a variation of FIG. 12.

### MODE FOR CARRYING OUT THE INVENTION

Hereinafter, the present invention will be described based on the following embodiments, however, the present invention is not limited by the following embodiments and can be altered in design within a scope in compliance with the intent described above and below, and all the changes are to be encompassed within a technical scope of the present invention. Note that, in each drawing, hatching, reference signs for components, and the like may be omitted for convenience of description, and in such a case, the specification and other drawings are to be referred to. Furthermore, since the dimensions of the various components in the drawings are provided for the purpose of facilitating the understanding of the feature of the present invention, the dimensions may differ from the actual dimensions in some cases.

### 1. Balloon for Balloon Catheter

A balloon for a balloon catheter in accordance with embodiments of the present invention is a balloon for a balloon catheter having a longitudinal axis direction and a radial direction, having a balloon body having an outer surface and an inner surface; and a protrusion part that projects outward from the outer surface of the balloon body in the radial direction and extends in the longitudinal axis direction, wherein the protrusion part comprises a tip region including an end tip in the radial direction and a base region located inward from the tip region in the radial direction in a cross-section perpendicular to the longitudinal axis direction; the balloon body and the protrusion part are composed of the same material; and a surface roughness of the base region is smaller than a surface roughness of the tip region where a surface roughness of the protrusion part is measured for a reference length in a direction perpendicular to the longitudinal axis direction.

Dilation of the stenosis with a balloon catheter is performed by delivering the balloon, which is provided at the distal end part of the balloon catheter, to the stenosis and then inflating the balloon to incise the stenosis by allowing the protrusion part disposed on the outer surface of the balloon body to bite into the stenosis. At this time, when the protrusion part enters the stenosis as the balloon inflates, the resistance to the pushing of the protrusion part into the stenosis increases in the final stage where the base region of the protrusion part enters the stenosis, making it difficult for the protrusion part to penetrate further. However, since the surface roughness of the base region is smaller than that of the tip region when the surface roughness is measured in the direction perpendicular to the longitudinal axis direction, the friction resistance of the base region is reduced, allowing the entire protrusion part to enter the stenosis. In addition, since the surface roughness of the tip region is greater than that of the base region, even if the protrusion part is inserted into a slippery lesion, it is less likely to deviate from the intended position, making it easier to incise the intended area.

In this specification, the balloon for a balloon catheter may simply be referred to as “the balloon.”

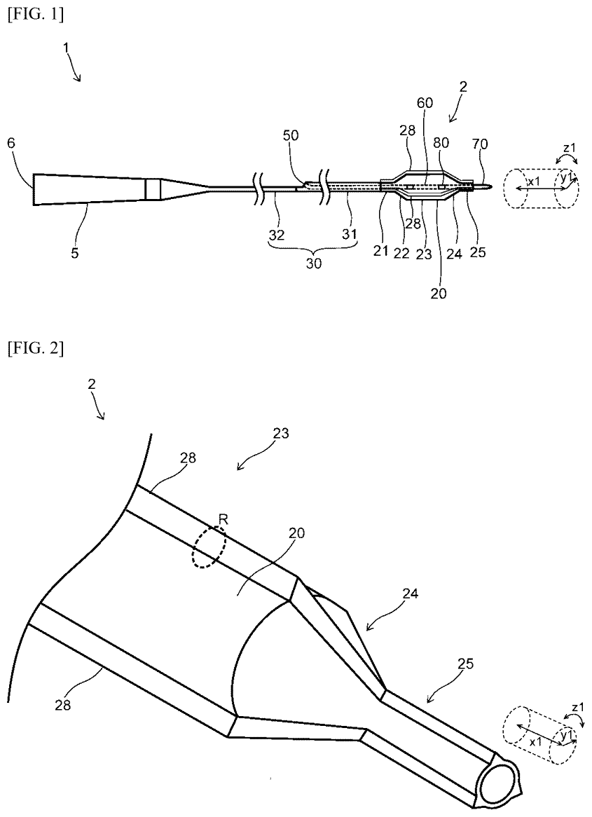

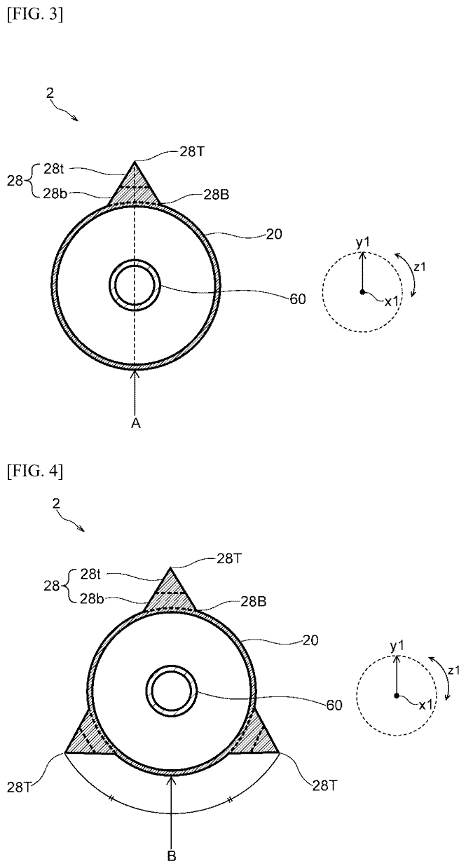

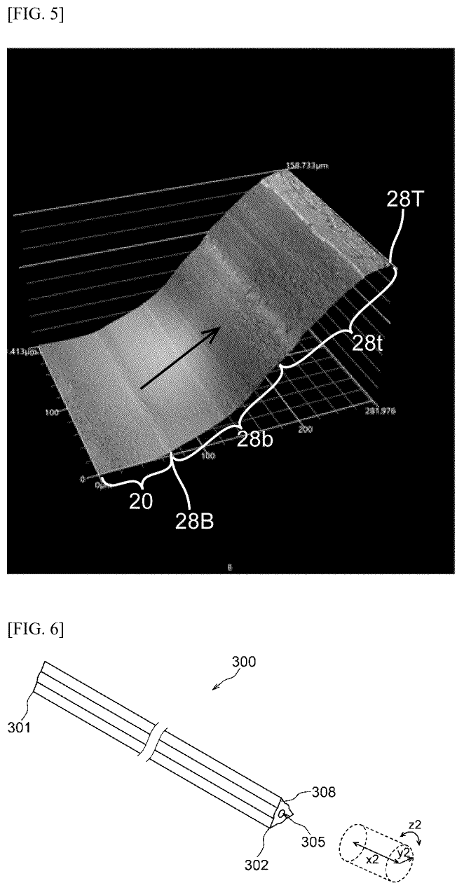

Hereinafter, a balloon for a balloon catheter in accordance with embodiments of the present invention will be described, referring to FIG. 1 to FIG. 5. FIG. 1 is a side view of a balloon catheter in accordance with one embodiment of the present invention. FIG. 2 is a perspective view of a balloon for a balloon catheter in accordance with one embodiment of the present invention, showing the distal side of the balloon. FIG. 3 is a cross-sectional view perpendicular to the longitudinal axis direction of a balloon for a balloon catheter in accordance with one embodiment of the present invention, and FIG. 4 is a cross-sectional view showing a variation of FIG. 3. FIG. 5 is an example of an image obtained when the surface roughness of a protrusion part of a balloon for a balloon catheter in accordance with an embodiment of the present invention is measured using a laser microscope.

As shown in FIG. 1, a balloon 2 is used for a balloon catheter 1. The balloon 2 is connected to a distal end part of a distal shaft 31, and the balloon 2 can be inflated by introducing a fluid through a lumen of the distal shaft 31 and the balloon 2 can be deflated by discharging the fluid. To control the inflation and deflation of the balloon 2, the fluid can be introduced or discharged using an indeflator (pressurizer for a balloon). The fluid may be a pressurized fluid that is pressurized by a pump or the like. The balloon catheter 1 will be described in detail in the section “2. balloon catheter.”

The balloon 2 has a longitudinal axis direction x1, a circumferential direction z1 along the outer edge of the balloon 2 in a cross-section perpendicular to the longitudinal axis direction x1, and a radial direction y1 connecting the figure center of the outer edge of the balloon 2 and a point on the outer edge of the balloon 2 in a cross-section perpendicular to the longitudinal axis direction x1. In this specification, the direction of the user's hand side in the longitudinal axis direction x1 is referred to as a proximal side, and the direction opposite the proximal side, i.e., the direction of the treatment target side, is referred to as a distal side.

The components and parts other than the balloon 2 have their own longitudinal axis direction, radial direction, and circumferential direction, which may be the same as or different from the longitudinal axis direction x1, radial direction y1, and circumferential direction z1 of the balloon 2. In this specification, for the sake of ease of understanding, all components and parts are described as having the same longitudinal axis direction, radial direction, and circumferential direction as the longitudinal axis direction x1, radial direction y1, and circumferential direction z1 of the balloon 2.

The balloon 2 has a proximal end and distal end in the longitudinal axis direction x1, and as shown in FIG. 1, preferably has a straight tubular part 23, a proximal tapered part 22 located proximal to the straight tubular part 23, a proximal sleeve part 21 located proximal to the proximal tapered part 22, a distal tapered part 24 located distal to the straight tubular part, and a distal sleeve part 25 located distal to the distal tapered part 24. The straight tubular part 23 preferably has a roughly cylindrical shape having approximately the same diameter in the longitudinal axis direction x1, but it may have a different diameter in the longitudinal axis direction x1. The proximal tapered part 22 and the distal tapered part 24 are preferably formed into a conical shape by reducing the diameter as they are apart from the straight tubular part 23. Since the straight tubular part 23 has the largest diameter, when the balloon 2 is inflated at a lesion site, such as a stenosis, the straight tubular part 23 can make sufficient contact with the lesion, make it easier to perform treatments such as dilation of the lesion. In addition, since the proximal tapered part 22 and the distal tapered part 24 are reduced in diameter, when the balloon 2 is deflated, the outer diameter of the proximal and distal end parts of the balloon 2 can be decreased, reducing the step between the distal shaft 31 and the balloon 2, thereby facilitating the insertion of the balloon 2 into the body cavity.

Preferably, while the proximal tapered part 22, the straight tubular part 23, and the distal tapered part 24 are the parts that inflate when a fluid is introduced into the balloon 2, the proximal sleeve part 21 and the distal sleeve part 25 are not inflated. At least part of the proximal sleeve part 21, which does not inflate, can be fixed to the distal shaft 31, and at least part of distal sleeve part 25 can be fixed to an inner shaft 60.

The balloon 2 has a balloon body 20 having an outer surface and an inner surface, and a protrusion part 28 that projects outward from the outer surface of the balloon body 20 in the radial direction y1 and extends in the longitudinal axis direction x1.

The balloon body 20 defines the basic shape of the balloon 2, and the protrusion part 28 is provided on the outer surface of the balloon body 20 in any pattern, such as linear, dot, mesh, or spiral patterns. By providing the protrusion part 28 on the outer surface of the balloon body 20, the protrusion part 28 can be provided with a scoring function, and it becomes possible to crack and dilate calcified stenotic areas in angioplasty. In addition, the protrusion part 28 can also contribute to improving the strength of the balloon 2 and preventing over-inflation during being pressurized.

The protrusion part 28 is preferably provided in the straight tubular part 23. This allows the protrusion part 28, which is provided in the straight tubular part 23 that most easily contacts the lesion, to more effectively dilate the stenosis area.

As shown in FIG. 1 and FIG. 2, the protrusion part 28 may be provided in the entire area of the balloon 2 in the longitudinal axis direction x1, i.e., in the straight tubular part 23. tapered parts, and sleeve parts. By providing the protrusion part 28 in portions other than the straight tubular part 23, the strength of the balloon 2 can be enhanced, and the effect of suppressing over-inflation during being pressurized can be improved. Alternatively, although not shown in the figures, the protrusion part 28 may be provided in the straight tubular part 23, while either not being provided in the tapered and sleeve parts or being provided at a lower height than in the straight tubular part 23. It also may be provided in at least a part of the tapered and sleeve parts. By having areas in the tapered and sleeve parts where the protrusion part 28 is either absent or provided at a lower height than in the straight tubular part 23, the insertion performance of the balloon 2 can be improved.

The protrusion part 28 is preferably made of the same material as the balloon body 20, and more preferably, the balloon body 20 and the protrusion part 28 are integrally molded. By forming the balloon body 20 and the protrusion part 28 from the same material, it becomes possible to prevent the protrusion part 28 from damaging the outer surface of the balloon body 20 while maintaining the flexibility of the balloon 2. By integrally forming the balloon body 20 and the protrusion part 28, detachment of the protrusion part 28 from the balloon body 20 can be prevented. Alternatively, when there is a certain degree of compatibility with the material used to form the balloon body 20, the material for forming the protrusion part 28 may differ from that of the balloon body 20. Such a balloon 2 can be produced, for example, by placing a parison obtained through extrusion molding in a mold with a groove and then blow-molding it. The preferred producing methods for the balloon will be described in the sections “3. First method for producing balloon catheter” and “4. Second method for producing balloon catheter.”

The balloon 2 may have an inner protrusion part that projects inward from the inner surface of the balloon body 20 in the radial direction y1. The protrusion part 28 and the inner protrusion part are preferably provided in the same position in the circumferential direction z1. The inner protrusion part is preferably integrally formed with the balloon body 20 and the protrusion part 28, and the inner protrusion part is preferably formed from the same material as the balloon body 20. Alternatively, when there is a certain degree of compatibility with the material used to form the balloon body 20, the material for forming the inner protrusion part may differ from that of the balloon body 20.

As shown in FIG. 3, the protrusion part 28 may be provided as a single protrusion part in the circumferential direction z1, or as shown in FIG. 4, the protrusion part 28 may be provided as a plurality of protrusions in the circumferential direction z1. When the balloon 2 has a plurality of protrusion parts 28 in the circumferential direction z1, each of the plurality of protrusion parts 28 is preferably spaced apart from each other in the circumferential direction z1, and more preferably arranged at equal intervals in the circumferential direction z1. The spacing distance is preferably longer than the maximum circumferential length of the protrusion part 28.

In the present invention, the protrusion part 28 is a portion that is formed to be thicker outward in the radial direction y1 than the film thickness at a predetermined position of the balloon body 20. The predetermined position refers to a position A, which is opposite in the circumferential direction z1 to an outer end 28T of the protrusion part 28 in the radial direction y1, when one protrusion part 28 is provided as shown in FIG. 3, and refers to a position B, which corresponds to the midpoint of the outer ends 28T in the circumferential direction z1 of the adjacent protrusion parts 28 in the circumferential direction z1, when a plurality of protrusion parts 28 are provided as shown in FIG. 4.

The maximum height of the protrusion part 28 in the radial direction y1 is preferably 1.2 times or more the film thickness at the predetermined position of the balloon body 20, more preferably 1.5 times or more, even more preferably 2 times or more, and may be 100 times or less, 50 times or less, 30 times or less, and 10 times or less is acceptable. The protrusion part 28 with a maximum height in the radial direction y1 in the above range allows for easier incision to an appropriate depth in the stenosis area, thereby facilitating crack formation.

The protrusion part 28 may have any cross-sectional shape in a cross-section perpendicular to the longitudinal axis direction x1, such as, for example, a triangle, a square, a polygon, a semi-circle, part of a circle, an approximate circle, a fan shape, a wedge shape, a convex shape, a spindle shape, and combinations thereof. Triangles, squares, and polygons shall include those with clear corner vertices and straight edges, as well as so-called rounded polygons with rounded corners and those with curved edges at least partly. Alternatively, the cross-sectional shape of the protrusion part 28 may be an irregular shape with concavity and convexity, chips, or the like.

When the protrusion part 28 is formed in a linear or dot shape, the protrusion part 28 is preferably arranged to extend along the longitudinal axis direction x1. Alternatively, the protrusion part 28 may be arranged to extend in a helical shape around the longitudinal axis.

As shown in FIG. 3 and FIG. 4, in a cross-section perpendicular to the longitudinal axis direction x1, the protrusion part 28 has an tip region 28t including the outer end 28T in the radial direction y1 and a base region 28b located inward from the tip region 28t in the radial direction y1, and a surface roughness of the base region 28b is smaller than a surface roughness of the tip region 28t where a surface roughness of the protrusion part 28 is measured for a reference length in a direction perpendicular to the longitudinal axis direction x1.

The protrusion part 28 can dilate the stenotic area by embedding into it to form cracks when the balloon 2 delivered to the stenotic area is inflated. At this time, the tip region 28t, which begins embedding into the stenotic area first, can do so with relatively low resistance to insertion, while the base region 28b, which embeds in the final stage, faces higher resistance to insertion, making it challenging for the entire protrusion part 28 to penetrate the stenotic area. However, since the surface roughness of the base region 28b is smaller than the surface roughness of the tip region 28t where the surface roughness in the direction perpendicular to the longitudinal axis direction x1 is measured, the frictional resistance of the base region 28b is reduced, allowing the entire protrusion part 28 to enter the stenotic area. This makes it easier to form cracks in the stenotic area, enabling efficient incision of the stenosis. Moreover, since the surface roughness of the tip region 28t is relatively greater than that of the base region 28b where the surface roughness in the direction perpendicular to the longitudinal axis direction x1 is measured, the protrusion part 28 is less likely to shift from the intended position even on slippery lesions, making it easier to incise the targeted area.

The surface roughness is the arithmetic mean roughness Ra of the roughness curve over a reference length, and the reference length is 100 μm. The arithmetic mean roughness Ra corresponds to the arithmetic mean roughness Ra as defined in JIS B 0601 (2001). For measurement, for example, a laser microscope with white light interferometry VK-X3000 manufactured by Keyence Corporation can be used. An example of an image obtained when measuring the surface roughness of the region R in FIG. 2 with the VK-X3000 is shown in FIG. 5. The arrow in FIG. 5 indicates the direction perpendicular to the longitudinal axis direction x1.

The surface roughness of the base region 28b can be obtained by measuring a roughness curve of 100 μm in the direction perpendicular to the longitudinal axis direction x1 from the base end 28B of the protrusion part 28 in the radial direction y1. The base end 28B of the protrusion part 28 is the part of the protrusion part 28 where the thickness in the radial direction y1 starts to exceed the film thickness at the above-described predetermined position of the balloon body 20.

The surface roughness of the tip region 28t can be obtained, for example, by measuring a roughness curve of 100 μm in the direction perpendicular to the longitudinal axis direction x1 from the outer end 28T. At this time, if the length from the base end 28B to the outer end 28T of the protrusion part 28 is shorter than twice the reference length of 100 μm, i.e., shorter than 200 μm, in the direction perpendicular to the longitudinal axis direction x1, the reference length of 100 μm from the outer end 28T in the direction perpendicular to the longitudinal axis direction x1 and the reference length of 100 μm from the base end 28B in the direction perpendicular to the longitudinal axis direction x1 must overlap in the direction perpendicular to the longitudinal axis direction x1, but the roughness of the tip region 28t and the base region 28b can be obtained by measuring the surface roughness by the above method.

Alternatively, the surface roughness of the tip region 28t can be obtained by measuring a roughness curve over a length of 100 μm in the direction perpendicular to the longitudinal axis direction x1, at a position on the outer end 28T side beyond a point located 100 μm from the base end 28B in the direction perpendicular to the longitudinal axis direction x1. At this time, if the length from the base end 28B to the outer end 28T of the protrusion part 28 is longer than twice the reference length of 100 μm, i.e., longer than 200 μm, in the direction perpendicular to the longitudinal axis direction x1, the reference length of 100 μm in the tip region 28t may either be a length from a point 100 μm from the base end 28B in the direction perpendicular to the longitudinal axis direction x1 to a point 100 μm toward the outer end 28T side, or a length of 100 μm in the direction perpendicular to the longitudinal axis direction x1 from the outer end 28T toward the base end 28B side. In this case, the surface roughness of the tip region 28t may be defined as the average of the surface roughness obtained from the roughness curve measured over the 100 μm reference length from a point 100 μm from the base end 28B to a point 100 μm toward the outer end 28T side, and the surface roughness obtained from the roughness curve measured over the 100 μm reference length from the outer end 28T toward the base end 28B side, both in the direction perpendicular to the longitudinal axis direction x1.

The surface roughness of each region is preferably determined as the average of the arithmetic mean roughness obtained from roughness curves measured over a specified number of reference lengths at predetermined intervals in the longitudinal axis direction x1. This allows for reducing the impact of surface roughness variations due to positional differences in the longitudinal axis direction x1. The predetermined interval may be, for example, 2 μm, and the specific number of reference lengths may be, for example, 31. In this case, the surface roughness over an area of 60 μm×100 μm can be obtained.

The surface roughness of each region may be compared at the same position in the longitudinal axis direction x1, or at different positions in the longitudinal axis direction x1, but they are preferably compared at the same position in the longitudinal axis direction x1. By having the surface roughness of the base region 28b smaller than the surface roughness of the tip region 28t at the same position in the longitudinal axis direction x1, it is possible to configure the protrusion part 28 to bite into a stenotic area more easily at the position. For example, the surface roughness of each region may be compared in the straight tubular part 23, and preferably the surface roughness of base region 28b is smaller than the surface roughness of the tip region 28t in the straight tubular part 23. When a plurality of protrusion parts 28 are provided, it suffices to measure the surface roughness of any one of the plurality of protrusion parts 28.

The surface roughness of the base region 28b is preferably 0.9 times or less the surface roughness of the tip region 28t, more preferably 0.8 times or less, even more preferably 0.6 times or less, may be 0.5 times or less, 0.4 times or less, and preferably 0.01 times or more, more preferably 0.05 times or more, and even more preferably 0.1 times or more, With the surface roughness within the above range, the entire protrusion part 28 can easily bite into a stenotic area, enabling the balloon 2 to more easily incise the intended site.

When a plurality of protrusion parts 28 are provided as shown in FIG. 4, it is preferable that the surface roughness of the base region 28b is smaller than that of the tip region 28t in all protrusion parts 28. This allows for easier incision of the stenotic area.

Materials forming the balloon body 20 and the protrusion part 28 include, for example, polyolefin-based resin such as polyethylene, polypropylene, ethylene-propylene copolymer; polyester-based resin such as polyethylene terephthalate and polyester elastomer; polyurethane-based resin such as polyurethane and polyurethane elastomer; polyphenylene sulfide-based resin; polyamide-based resin such as polyamide and polyamide elastomer; fluorine-based resin; silicone-based resin; and natural rubber such as latex rubber. Only one of these may be used, or two or more may be used in combination. Of these, polyamide-based resin, polyester-based resin, and polyurethane-based resin are preferably used, more preferably polyamide-based resin such as nylon 12 and nylon 11, and particularly preferably nylon 12. From the viewpoint of thinning and flexibility of the balloon body 20, elastomer resin is preferably used, and polyamide elastomer such as polyamide ether elastomer is preferably used.

A surface roughness of the balloon body 20 is preferably larger than the surface roughness of the base region 28b where the surface roughness of the protrusion part 28 and the surface roughness of the balloon body 20 are measured for a reference length in a direction perpendicular to the longitudinal axis direction x1. While the balloon 2 in accordance with embodiments of the present invention has a lower surface roughness of the base region 28b, which allows the entire protrusion part 28 to enter the stenotic area, the greater surface roughness of the balloon body 20 than that of the base region 28b can prevents the balloon body 20 itself from entering the stenotic area, thereby keeping the crack from widening excessively.

The surface roughness of the balloon body 20 is preferably 1.2 times or more the surface roughness of the base region 28b, more preferably 1.5 times or more, even more preferably 2 times or more, particularly preferably 3 times or more, and preferably 10 times or less, more preferably 9 times or less, and even more preferably 8 times or less.

The surface roughness of the balloon body 20 is preferably larger than the surface roughness of the tip region 28t where the surface roughness of the protrusion part 28 and the surface roughness of the balloon body 20 are measured for a reference length in a direction perpendicular to the longitudinal axis direction x1. This makes it possible to establish a relationship between the surface roughness of the balloon body 20 and each region of the protrusion part 28 to be set as “base region 28b<tip region 28t<balloon body 20”. With the balloon body 20, which has the largest area in contact with the blood vessel wall, having the greatest surface roughness when measured in the direction perpendicular to the longitudinal axis direction x1, the resistance of the balloon body 20 against the blood vessel wall can be increased, making it easier to fix the balloon 2 in the desired position. This makes it possible to fix the balloon 2 in the desired position and perform treatment.

The surface roughness of the balloon body 20 is preferably 1.05 times or more the surface roughness of the tip region 28t, more preferably 1.1 times or more, even more preferably 1.2 times or more, particularly preferably 1.5 times or more, and preferably 8 times or less, more preferably 7 times or less, and even more preferably 6 times or less.

### 2. Balloon Catheter

A balloon catheter 1 in accordance with embodiments of the present invention is provided with the above-described balloon 2 for a balloon catheter. As described in the section “1. Balloon for balloon catheter,” the balloon 2 is connected to the distal end part of the distal shaft 31, as shown in FIG. 1.

FIG. 1 illustrates a so-called rapid-exchange type balloon catheter 1, which has a guidewire port 50 located midway between the distal and proximal sides of the shaft 30 and has an inner shaft 60 that functions as a guidewire lumen from the guidewire port 50 to the distal side of the shaft 30. The balloon catheter 1 preferably has a distal shaft 31 and a proximal shaft 32, with the distal shaft 31 and the proximal shaft 32 being separate members. The proximal end part of the distal shaft 31 may be connected to the distal end part of the proximal shaft 32, thereby forming the shaft 30 that extends from the balloon 2 to the proximal end part of the balloon catheter 1. Alternatively, a single shaft 30 may extend from the balloon 2 to the proximal end part of the balloon catheter 1, or the distal shaft 31 and the proximal shaft 32 may each consist of multiple tube members.

The shaft 30 preferably has a fluid flow path and a guidewire lumen inside. To configure the shaft 30 to have the internal fluid flow path and the guidewire lumen, for example, the inner shaft 60 located inside the shaft 30 may function as the guidewire lumen, and the space between the shaft 30 and the inner shaft 60 may function as the fluid flow path. In such a configuration, preferably, the inner shaft 60 extends from the distal end of the shaft 30 and passes through the balloon 2, the distal side of the balloon 2 is connected to the inner shaft 60, and the proximal side of the balloon 2 is connected to the shaft 30.

The shaft 30 is preferably composed of resin, metal, or a combination of resin and metal. By using resin as the material for the shaft, flexibility and elasticity can be more easily imparted to the shaft 30. By using metal as the material for the shaft 30, the delivering performance of the balloon catheter 1 can be improved. Examples of resin used for the shaft 30 include polyamide-based resin, polyester-based resin, polyurethane-based resin, polyolefin-based resin, fluorine-based resin, polyvinyl chloride-based resin, silicone-based resin, natural rubber, and synthetic rubber. Any one of these may be used alone, or two or more may be used in combination. Examples of metal used for the shaft 30, include stainless steel such as SUS 304 and SUS 316, platinum, nickel, cobalt, chromium, titanium, tungsten, gold, Ni-Ti alloys, Co-Cr alloys, or combinations thereof. When the shaft 30 is composed of the distal shaft 31 and proximal shaft 32 as separate members, the distal shaft 31 may be, for example, made of resin, and the proximal shaft 32 may be made of metal. The shaft 30 may also have a layered structure using different materials or the same material.

The balloon 2 and the shaft 30 may be joined by adhesive bonding, welding, or by attaching a ring-shaped member at the point where the end of the balloon 2 and the shaft 30 overlap to swage them. Of these, the balloon 2 and the shaft 30 are preferably joined by welding. By welding the balloon 2 and the shaft 30, the bond between the balloon 2 and the shaft 30 is difficult to be released even when the balloon 2 is repeatedly inflated and deflated, easily increasing the strength of the bond between them.

The balloon catheter 1 is preferably provided with a tip member 70 at its distal end part. The tip member 70 may be provided at the distal end part of the balloon catheter 1 by being connected to the distal end part of the balloon 2 as a separate component from the inner shaft 60, or the inner shaft 60 extending distally beyond the distal end of the balloon 2 may function as the tip member 70.

A radiopaque marker 80 may be placed on the inner shaft 60 inside the balloon 2 at the location of the balloon 2 in the longitudinal axis direction x1, so that the position of the balloon 2 can be confirmed radiographically. The radiopaque marker 80 is preferably placed at a position corresponding to both ends of the straight tubular part 23 of the balloon 2, or may be placed at a position corresponding to the center of the straight tubular part 23 in the longitudinal axis direction x1.

A hub 5 may be provided at a proximal side of the shaft 30, and the hub 5 may be provided with a fluid inlet 6 that is connected to the flow channel of the fluid supplied to the interior of the balloon 2.

The shaft 30 and the hub 5 may be joined by, for example, adhesive bonding or welding. Of these, the shaft 30 and the hub 5 are preferably joined by adhesive bonding. The adhesive bonding of the shaft 30 and hub 5 can increase the bonding strength of the shaft 30 and hub 5 to increase durability of the balloon catheter 1 when the materials forming the shaft 30 and hub 5 are different, for example, in a case where the shaft 30 is made of material having high flexibility and the hub 5 is made of material having high stiffness.

Although not shown in the figures, the present invention is also applicable to a so-called over-the-wire type balloon catheter that has a guidewire lumen extending from the distal end to the proximal end of the shaft. In the case of the over-the-wire type, the inflation lumen and the guidewire lumen preferably extend to a hub positioned at the proximal side, and the proximal openings of each lumen are preferably provided in the hub having a bifurcated structure.

In the case of the rapid-exchange type catheter, the outer wall of the distal shaft 31 and/or the proximal shaft 32 is preferably coated as appropriate, and more preferably, both the distal shaft 31 and the proximal shaft 32 are coated. In the case of the over-the-wire type catheter, the outer wall of the outer shaft is preferably coated as appropriate.

The coating can be a hydrophilic or hydrophobic coating, depending on the purpose, and can be applied by dipping the shaft 30 into a hydrophilic or hydrophobic coating agent, applying a hydrophilic or hydrophobic coating agent to the outer wall of the shaft 30, or coating the outer wall of the shaft 30 with a hydrophilic or hydrophobic coating agent. The coating agent may contain medical agents and additives.

Hydrophilic coating agents include hydrophilic polymers such as polyvinyl alcohol, polyethylene glycol, polyacrylamide, polyvinyl pyrrolidone, methyl vinyl ether maleic anhydride copolymer, and hydrophilic coating agents made of any combination thereof.

Hydrophobic coating agents include polytetrafluoroethylene (PTFE), ethylene-propylene fluoride (FEP), perfluoroalkoxy alkane (PFA), silicone oil, hydrophobic urethane resin, carbon coat, diamond coat, diamond-like carbon (DLC) coating, ceramic coating, and substances with low surface free energy terminated with an alkyl group or a perfluoroalkyl group.

### 3. First Method for Producing Balloon Catheter

A first method for producing a balloon catheter in accordance with embodiments of the present invention is a method for producing the above balloon catheter, and has a step of preparing a parison having a tubular shape and composed of resin; a step of preparing a mold having a longitudinal axis direction and a radial direction, and having an inner cavity extending in the longitudinal axis direction through which the parison is to be inserted, the inner cavity consisting of a groove part recessed outwardly in the radial direction and extending in the longitudinal axis direction and a cylindrical wall part other than the groove part; a step of inserting a cathode conductive member having an outer shape along the cylindrical wall part in a cross-section in the longitudinal axis direction into the inner cavity of the mold to bring the cathode conductive member into contact with the cylindrical wall part without contacting the groove part; a step of connecting the cathode conductive member to a cathode of a power source and connecting an outer wall of the mold to an anode of the power source; a step of introducing an electrolytic solution into the inner cavity of the mold, outside the cathode conductive member in the radial direction; a step of supplying power from the power source to perform electrochemical polishing; a step of removing the electrolytic solution and removing the cathode conductive member from the inner cavity of the mold, followed by inserting the parison into the inner cavity of the mold; and a step of introducing a fluid into a lumen of the parison to inflate the parison and bringing an outer wall of the parison into contact with the groove part and the cylindrical wall part of the mold, wherein in a cross-section perpendicular to the longitudinal axis direction, the groove part has a groove tip region with a longer distance from a figure center of the inner cavity of the mold in the radial direction and a groove base region with a shorter distance from the figure center in the radial direction than that of the groove tip region.

By bringing the cathode conductive member into contact with the cylindrical wall part of the mold without allowing it to contact the groove part of the mold, introducing the electrolytic solution into the inner cavity of the mold, outside the cathode conductive member in the radial direction, and connecting the cathode conductive member to the cathode of the power source and connecting the outer wall of the mold to the anode of the power source, it is possible to electrochemically polish only the groove part of the mold without electrochemically polishing the cylindrical wall part of the mold. Since the groove part of the mold has the groove tip region with a longer distance from the figure center of the inner cavity of the mold in the radial direction and the groove base region with a shorter distance from the figure center of the mold in the radial direction than that of the groove tip region, the groove tip region is farther from the cathode conductive member, and the groove base region is closer to the cathode conductive member. Therefore, in the groove part of the mold, the groove tip region is less susceptible to electrochemical polishing, while the groove base region is more susceptible to electrochemically polishing, allowing for the preparation of the mold with the groove tip region having a greater surface roughness and the groove base region with a smaller surface roughness. By placing the parison in the inner cavity of this mold and inflating it, a shape with greater surface roughness is transferred to the part of the parison that contacts the groove tip region, and a shape with smaller surface roughness is transferred to the part that contacts the groove base region. This makes it possible to produce “2. Balloon catheter” that has “1. Balloon for balloon catheter” with a surface roughness of the base region that is smaller than the surface roughness of the tip region.

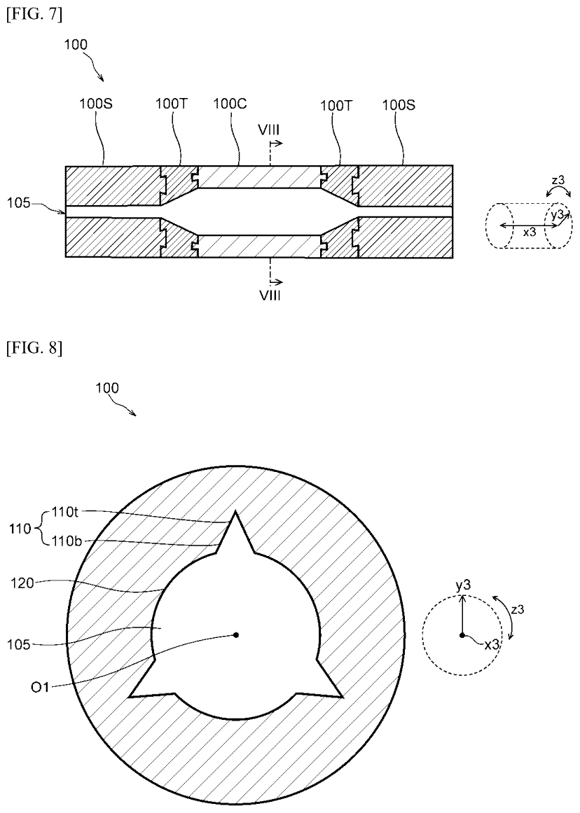

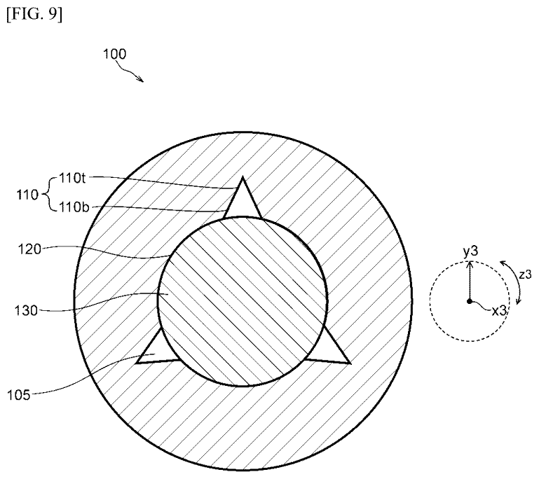

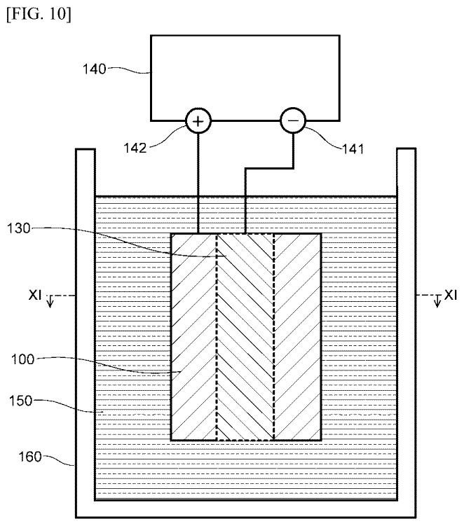

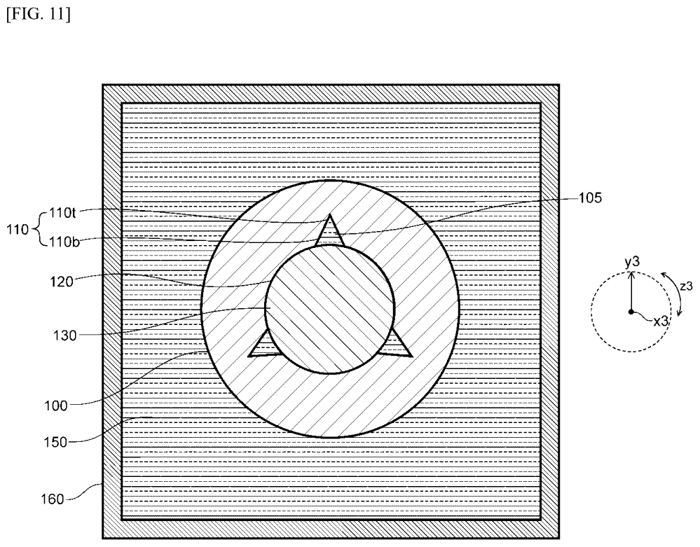

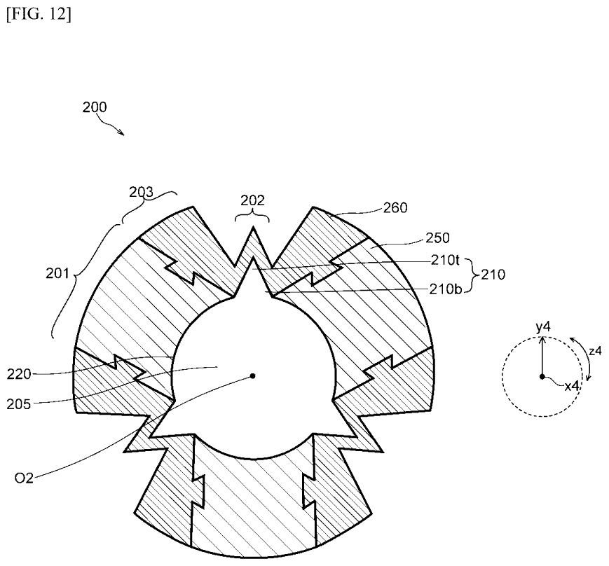

Referring to FIG. 6 to FIG. 11, the first method for producing the balloon catheter in accordance with embodiments of the present invention is explained. FIG. 6 is a perspective view of a parison in accordance with one embodiment of the present invention before inflation. FIG. 7 is a cross-sectional view perpendicular to the longitudinal axis direction of a first mold used in a first producing method in accordance with one embodiment of the present invention. FIG. 8 is a VIII-VIII cross-sectional view of the first mold shown in FIG. 7. FIG. 9 is a cross-sectional view perpendicular to the longitudinal axis direction when a cathode conductive member is inserted into an inner cavity of the first mold shown in FIG. 8. FIG. 10 is a schematic view explaining the first method in accordance with one embodiment of the present invention. FIG. 11 is a XI-XI cross-sectional view of FIG. 10.

First, a parison 300 is prepared. As shown in FIG. 6, the parison 300 is a tubular member made of resin and having a lumen 305. The parison 300 is made, for example, by extrusion molding. The parison 300 has a first end 301 and a second end 302, and extends in a longitudinal axis direction x2 from the first end 301 to second end 302. The parison 300 has a radial direction y2 and a circumferential direction z2, similar to the balloon 2.

The cross-sectional shape perpendicular to the longitudinal axis direction x2 of the parison 300 may be uniform in the longitudinal axis direction x2. This improves the productivity of the parison 300. Alternatively, the cross-sectional shape perpendicular to the longitudinal axis direction x2 of the parison 300 may differ depending on the position in the longitudinal axis direction x2. For example, the outer diameter of a portion of the parison 300 in the longitudinal axis direction x2 may be larger than that of other portions, and the portion with the larger outer diameter may be molded to become the straight tubular part 23 of the balloon 2. In order to manufacture a parison 300 with an outer diameter that differs depending on the position in the longitudinal axis direction x2, blow molding may be performed using a mold in advance.

As shown in FIG. 6, the parison 300 before inflation may have a protrusion part 308 that is thicker outwardly in the radial direction y2. By making the protrusion part 308 contact a groove part 110 of a first mold 100 or a groove part 210 of a second mold 200, which are described below, it becomes easier to form the protrusion part 28 of the balloon 2.

As shown in FIG. 6, the protrusion parts 308 may be provided in multiple locations in the circumferential direction z2, or, although not shown in the figures, the protrusion part 308 may be provided in a single location in the circumferential direction z2. In the case where the protrusion parts 308 are provided in multiple locations in the circumferential direction z2, the multiple protrusion parts 308 are preferably space apart from each other in the circumferential direction z2, and more preferably arranged at equal intervals in the circumferential direction z2.

As for the materials constituting the parison 300, reference can be made to the description of the resin used for forming the balloon body 20 and the protrusion part 28 in the section “1. Balloon for balloon catheter.”

As shown in FIG. 7, the first mold 100 used for the first producing method of the present invention has a longitudinal axis direction x3, a radial direction y3, and a circumferential direction z3, and has an inner cavity 105 extending in the longitudinal axis direction x3 through which the parison 300 is to be inserted. Preferably, a part of the parison 300 in the longitudinal axis direction x2 is positioned within the inner cavity 105 of the first mold 100. The longitudinal axis direction x2 of the parison 300 preferably aligns with the longitudinal axis direction x3 of the first mold 100. This facilitates the placement of the parison 300 within the inner cavity 105 of the first mold 100.

The first mold 100 preferably has, in the longitudinal axis direction x3, a first mold straight tubular part 100C, which forms the straight tubular part 23 of the balloon 2, two first mold tapered parts 100T, positioned on both sides of the first mold straight tubular part 100C to form the tapered parts of the balloon 2, and two first mold sleeve parts 100S, positioned further side of the first mold straight tubular part 100C than the first mold tapered parts 100T to form the sleeve parts of the balloon 2. This allows the first mold straight tubular part 100C to form the straight tubular part 23 of the balloon 2, the first mold tapered parts 100T to form the proximal tapered part 22 and distal tapered part 24 of the balloon 2, and the first mold sleeve parts 100S to form the proximal sleeve part 21 and distal sleeve part 25 of the balloon 2.

The first mold 100 may be composed of a single component or multiple components. As shown in FIG. 7, it may be configured by connecting multiple mold components along the longitudinal axis direction x3. For example, the first mold straight tubular part 100C, the first mold tapered parts 100T, and the first mold sleeve parts 100S may each be separate mold components, and they may be connected to each other along the longitudinal axis direction x3. In addition, the first mold 100 may be divisible in the radial direction y3. This makes it easier to insert a cathode conductive member 130, which will be described later, into the inner cavity 105 of the first mold 100. As shown in FIG. 7, each mold component may be joined by engaging adjacent mold components with each other, or though not shown in the figures, a magnet may be attached to each adjacent mold component to enable joining through magnetic attraction.

As shown in FIG. 8, the inner cavity 105 of the first mold 100 consists of a groove part 110 recessed outwardly in the radial direction y3 and extending in the longitudinal axis direction x3 and a cylindrical wall part 120 other than the groove part 110. After the step of electrochemical polishing, which will be described later, the parison 300 is inserted into the inner cavity 105 of the first mold 100, and a fluid is then introduced into the lumen 305 of the parison 300 to inflate the parison 300. At this time, the parison 300 enters the groove part 110, and the portion that enters the groove part 110 is formed into the protrusion part 28 of the balloon 2, while the portion that contacts the cylindrical wall part 120 is formed into the balloon body 20. This process allows the formation of the balloon 2, which has the balloon body 20 and the protrusion part 28, from the parison 300.

As shown in FIG. 8, multiple groove parts 110 may be provided in the circumferential direction z3, or, although not shown in the figures, a single groove part 110 may be provided in the circumferential direction z3. In the case where multiple groove parts 110 is provided in the circumferential direction z3, the groove parts 110 are preferably spaced apart from each other in the circumferential direction z3, and more preferably, they are arranged at equal intervals in the circumferential direction z3.

In a cross-section perpendicular to the longitudinal axis direction x3, the groove part 110 has a groove tip region 110t with a longer distance from a figure center O1 of the inner cavity 105 of the first mold 100 in the radial direction y3 and a groove base region 110b with a shorter distance from the figure center O1 in the radial direction y3 than that of the groove tip region 110t. The tip region 28t of the balloon 2 can be formed by the groove tip region 110t, and the base region 28b of the balloon 2 can be formed by the groove base region 110b.

Preferably, the groove part 110 is provided in the first mold straight tubular part 100C of the first mold 100. This allows the protrusion part 28 to be formed on the straight tubular part 23 of the balloon 2, thereby enhancing the efficiency of incising stenosis with the balloon 2.

The groove part 110 may be provided in at least one of the two first mold tapered parts 100T of the first mold 100. This allows the protrusion part 28 to be formed on the proximal tapered part 22 and/or distal tapered part 24 of the balloon 2, improving non-slip performance of the balloon 2 against the stenosis. When the groove part 110 is provided in the first mold tapered parts 110T, the depth of the groove part 110 provided in the first mold tapered parts 110T is preferably no greater than the depth of the groove part 110 provided in the first mold straight tubular part 100C. This allows the height of the protrusion part 28 formed on the proximal tapered part 22 and/or distal tapered part 24 of the balloon 2 to be no greater than the height of the protrusion part 28 formed on the straight tubular part 23, improving the insertion performance of the balloon 2. If the groove part 110 is not provided in the first mold tapered parts 100T, the protrusion part 28 will not be formed on the proximal tapered part 22 and/or distal tapered part 24 of the balloon 2, or the height of the protrusion part 28 will be reduced, further improving the insertion performance of the balloon 2. In this case, an inner protrusion part may be formed in the areas where the protrusion part 28 is either not formed or is formed with a reduced height.

The groove part 110 may or may not be provided in the first mold sleeve parts 100S of the first mold 100. When the groove part 110 is provided in the first mold sleeve parts 100S, the depth of the groove part 110 provided in the first mold sleeve parts 100S is preferably smaller than the depth of the groove part 110 provided in the first mold straight tubular part 100C. This allows the height of the protrusion part 28 formed on the proximal sleeve part 21 and/or distal sleeve part 25 to be lower than the protrusion part formed on the straight tubular part 23, improving the insertion performance of the balloon 2. If the groove part 110 is not provided in the first mold sleeve parts 100S, the protrusion part 28 will not be formed on the proximal sleeve part 21 and/or distal sleeve part 25 of the balloon 2, further improving the insertion performance of the balloon 2.

Prior to inserting the parison 300 into the inner cavity 105 of the first mold 100, the inner cavity 105 undergoes electrochemical polishing. In the process of electrochemical polishing, the cathode conductive member 130 is inserted into the inner cavity 105. As shown in FIG. 9, the cathode conductive member 130 has an outer diameter along the cylindrical wall part 120 in a cross-section of the longitudinal axis direction x3. When inserting the cathode conductive member 130 into the inner cavity 105 of the first mold 100, the cathode conductive member 130 is no in contact with the groove part 110 but is in contact with the cylindrical wall part 120. In other words, the cathode conductive member 130 has a region where its outer wall is along the cylindrical wall part 120 and a region where a gap is present between the outer wall of the cathode conductive member 130 and the wall of the cavity 105 of the first mold 100 due to the groove part 110. As a result, the cathode conductive member 130 is positioned closer to the groove base region 110b and farther from the groove tip region 110t.

The shape of the cathode conductive member 130 may be any shape in which its outer profile follows the cylindrical wall part 120 of the first mold 100, and it may be tubular or solid. In the region where a gap is present between the outer wall of the cathode conductive member 130 and the wall of the inner cavity 105 of the first mold 100 due to the groove part 110, the cathode conductive member 130, preferably, does not protrude in the radial direction y3. This allows the cathode conductive member 130 to be easily positioned closer to the groove base region 110b and farther from the groove tip region 110t.

As shown in FIG. 10. the cathode conductive member 130 is connected to a cathode 141 of a power source 140 and the outer wall of the first mold 100 is connected to an anode 142 of the power source 140. The first mold 100 is made of metal, and the first mold 100 functions as an anode.

As shown in FIG. 11, an electrolytic solution 150 is introduced into the inner cavity 105 of the first mold 100, outside the cathode conductive member 130, i.e., into the groove part 110. This step may be carried out, as shown in FIG. 10, by immersing the first mold 100, with the cathode conductive member 130 inserted into its inner cavity 105, in an electrolytic bath 160 filled with the electrolytic solution 150. In this setup, when power is supplied from the power source 140 to apply a voltage between the cathode conductive member 130 and the first mold 100 functioning as an anode, the metal elements on the surface of the inner cavity 105 of the first mold 100 functioning as an anode that is in contact with the electrolytic solution 150 dissolve into the electrolytic solution 150. As a result, the surface of the inner cavity 105 of the first mold 100 that is in contact with the electrolytic solution 150 is electrochemically polished.

Since the cathode conductive member 130 has the outer diameter along the cylindrical wall part 120, the cathode conductive member 130 is positioned farther from the groove tip region 110t and closer to the groove base region 110b. Accordingly, the anode-cathode distance between the cathode conductive member 130 and the groove base region 110b becomes shorter, resulting in a higher current density, which increases the amount of electrochemical polishing in the groove base region 110b. In contrast, the anode-cathode distance between the cathode conductive member 130 and the groove tip region 110t becomes longer, resulting in a lower current density, which leads to less electrochemical polishing in the groove tip region 110t than in the groove base region 110b. As a result, the inner cavity 105 of the first mold 100 is processed in such a way that the groove base region 110b is polished more smoothly, while the groove tip region 110t has a rougher surface.

After the electrochemical polishing, the electrolytic solution 150 is removed from the inner cavity 105 of the first mold 100. This step may be carried out by lifting the first mold 100, with the cathode conductive member 130 inserted into its inner cavity 105, out of the electrolytic bath 160.

After removing the cathode conductive member 130 from the inner cavity 105 of the first mold 100, the parison 300 is inserted into the inner cavity 105 of the first mold 100, and a fluid is introduced into the lumen 305 of the parison 300 to inflate the parison 300 and bring the outer wall of the parison 300 into contact with the groove part 110 and the cylindrical wall part 120 of the first mold 100. At this time, preferably, the parison 300 is blow molded by heating the first mold 100. The heating of the first mold 100 can be performed using known methods, such as heating with a heater disposed on the outer side of the first mold 100 or heating the first mold 100 itself via induction heating. These steps allow for the production of the balloon 2 with the protrusion part 28, where the surface roughness of the base region 28b, formed by the more smoothly polished groove base region 110b, is smaller, and the surface roughness of the tip region 28t, formed by the rougher surface of the groove tip region 110t, is larger.

In the electrochemical polishing step, the cathode conductive member 130 is brought into contact with the cylindrical wall part 120, making it difficult for the electrolytic solution 150 to penetrate between the cathode conductive member 130 and the cylindrical wall part 120. As a result, the surface of the cylindrical wall part 120 is less likely to undergo electrochemical polishing, allowing it to remain rough. This enables the production of the balloon 2 with a surface roughness of the balloon body 20 that is greater than the surface roughness of the protrusion part 28.

In the step of bringing the cathode conductive member 130 into contact with the cylindrical wall part 120, no gap is preferably formed between the cathode conductive member 130 and the cylindrical wall part 120. This ensures that the surface of the cylindrical wall part 120 is not electrochemically polished, making it easier to achieve a surface roughness of the balloon main body 20 that is greater than that of the protruding portion 28.

Materials constituting the first mold 100 is preferably metal, and more preferably, iron, copper, aluminum, or an alloy of these. Stainless steel is an example of an iron alloy, brass is an example of a copper alloy, and duralumin is an example of an aluminum alloy.

From the point of view of having sufficient conductivity and strength, as well as being easy to process, the first mold 100 is preferably made of stainless steel.

Materials constituting the cathode conductive member 130 is not particularly limited as long as it has sufficient conductivity, and metals such as stainless steel, titanium, copper, aluminum, platinum, gold, or their alloys can be mentioned as examples.

The electrolytic solution 150 is not particularly limited, and depending on the material constituting the first mold 100, it can be appropriately selected from known aqueous solutions, such as alcohol-based or sulfuric acid-based solutions. When using an electrolytic bath 160, the electrolytic bath 160 is preferably made of a material that is not corroded by the electrolytic solution 150.

### 4. Second Method for Producing Balloon Catheter

A second method for producing the balloon catheter in accordance with embodiments of the present invention is a method for producing the above balloon catheter, and has a step of preparing a parison having a tubular shape and composed of resin; a step of preparing a mold having a longitudinal axis direction and a radial direction, and having an inner cavity extending in the longitudinal axis direction through which the parison is to be inserted, the inner cavity consisting of a groove part recessed outwardly in the radial direction and extending in the longitudinal axis direction and a cylindrical wall part other than the groove part; a step of inserting the parison into the inner cavity of the mold; a step of heating the mold; and a step of introducing a fluid into a lumen of the parison to inflate the parison and bringing an outer wall of the parison into contact with the groove part and the cylindrical wall part of the mold, wherein in a cross-section perpendicular to the longitudinal axis direction, the groove part has a groove tip region with a longer distance from a figure center of the inner cavity of the mold in the radial direction and a groove base region with a shorter distance from the figure center in the radial direction than that of the groove tip region; in a cross-section perpendicular to the longitudinal axis direction, the mold has a first region with a low thermal conductivity, a second region with a higher thermal conductivity than the first region, and a third region with a higher thermal conductivity than the second region; and in a cross-section perpendicular to the longitudinal axis direction, the first region is located at the cylindrical wall part, the second region is located at the groove tip region, and the third region is located at the groove base region.

By positioning the second region at the groove tip region and the third region, which has a higher thermal conductivity than the second region, at the groove base region, when the parison is inserted into the inner cavity of the mold, the mold is heated, and the parison is inflated so that its outer wall contacts the inner cavity of the mold, the resin in the portion contacting the groove tip region, where the second region with lower thermal conductivity is located, is less likely to soften, resulting in the formation of a rougher surface in the tip region. In contrast, the resin in the portion contacting the groove base region, where the third region with higher thermal conductivity is located, softens more easily, allowing the formation of a smoother surface in the base region. In addition, since the first region, which has a lower thermal conductivity than the second region, is located in the cylindrical wall part of the mold, the surface roughness of the balloon body formed by the cylindrical wall part can be increased.

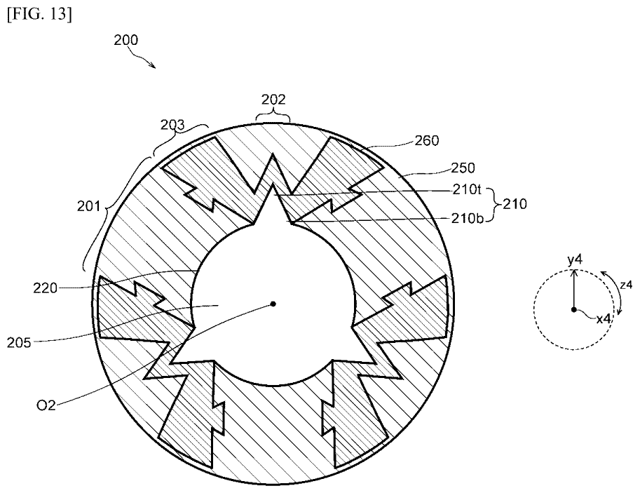

Referring to FIG. 12 and FIG. 13, the second method for producing the balloon catheter in accordance with embodiments of the present invention is explained. FIG. 12 is a cross-sectional view perpendicular to the longitudinal axis direction of a second mold used in a second producing method in accordance with one embodiment of the present invention, FIG. 13 is a cross-sectional view showing a variation of FIG. 12.

First, a parison is prepared. The parison used in the second producing method is the same as the parison 300 used in the above-described first producing method, and the description of the parison 300 in the section “3. First method for producing balloon catheter” and FIG. 6 can be referenced. Hereinafter, the parison used in the second producing method will be explained used the same reference sign, with reference to FIG. 6.

Next, a second mold 200 used in the second producing method is prepared. The second mold 200 has a longitudinal axis direction x4, a radial direction y4, and circumferential direction z4, and has an inner cavity 205 extending in the longitudinal axis direction x4 through which the parison 300 is inserted, and the inner cavity 205 consists of a groove part 210 recessed outwardly in the radial direction y4 and extending in the longitudinal axis direction x4 and a cylindrical wall part 220 other than the groove part 210.

In a cross-section perpendicular to the longitudinal axis direction x4, the groove part 210 has a groove tip region 210t with a longer distance from a figure center O2 of the inner cavity 205 of the second mold 200 in the radial direction y4 and a groove base region 210b with a shorter distance from the figure center O2 in the radial direction y4 than that of the groove tip region 210t.

The groove part 210, groove tip region 210t, and groove base region 210b of the second mold 200 are similar to the groove part 110, groove tip region 110t, and groove base region 110b of the first mold 100, and the description in the section “3. First method for producing balloon catheter” can be referenced. Regarding the second mold 200, except for the details described below, the second mold 200 is the same as the first mold 100, and the description in the above section “3. First method for producing balloon catheter” and FIG. 7 can be referenced.

As shown in FIG. 12 and FIG. 13, in a cross-section perpendicular to the longitudinal axis direction x4, the second mold 200 has a first region 201 with a low thermal conductivity, a second region 202 with a higher thermal conductivity than the first region 201, and a third region 203 with a higher thermal conductivity than the second region 202, and the first region 201 is located at the cylindrical wall part 220, the second region 202 is located at the groove tip region 210t, and the third region 203 is located at the groove base region 210b.

The parison 300 is inserted into the inner cavity 205 of the second mold 200 having such a configuration, the second mold 200 is heated, and a fluid is introduced into the lumen 305 of the parison 300 to inflate the parison 300, so that the outer wall of the parison 300 comes into contact with the groove part 210 and the cylindrical wall part 220 of the second mold 200. At this time, preferably, the parison 300 is blow molded by heating the second mold 200. The heating of the second mold 200 can be performed using known methods, such as heating with a heater disposed on the outer side of the second mold 200 or heating the second mold 200 itself via induction heating. Through these steps, the parison 300 in contact with the groove base region 210b, where the third region 203 with high thermal conductivity is located, is heated to a higher temperature, so that the resin becomes smoother and the surface roughness is reduced. In contrast, the parison 300 in contact with the groove tip region 210t, where the second region 202 with lower thermal conductivity than the third region 203 is located, is heated to a lower temperature than the parison 300 in the groove base region 210b, so that the smoothing of the resin is suppressed. As a result, the balloon 2 can be produced in which the surface roughness of the base region 28b is smaller than that of the tip region 28t.

Since the first region 201, having the lowest thermal conductivity, is located in the cylindrical wall part 220 of the second mold 200, the parison 300 in contact with the cylindrical wall part 220 is heated to an even lower temperature, so that the smoothing of the resin is further suppressed. As a result, the balloon 2 can be produced in which the surface roughness of the balloon body 20 is greater than that of the protrusion part 28.

The thermal conductivity of the third region 203 may be configured to be higher than that of the second region 202 by the following arrangement: the first region 201 is composed of a first member 250 formed from a material (L) with low thermal conductivity, while the second region 202 and the third region 203 include a second member 260 formed from a material (H) with higher thermal conductivity than the material (L); and the thickness in the radial direction y4 of the second member 260 in the third region 203 is greater than the thickness in the radial direction y4 of the second member 260 in the second region 202.

As shown in FIG. 12, the second mold 200 may be configured so that the second member 260, which has a thin thickness in the second region 202 and a thick thickness in the third region 203, and the first member 250, which has a thickness equivalent to that of the second member 260 in the third region 203, are connected in the circumferential direction z4. In the case where multiple grooves 210 are provided in the circumferential direction z4, it is preferable that the second member 260 is also arranged in accordance with the position of the grooves 210 in the circumferential direction z4. In this case, in the second region 202, there is usually air outside the radial direction y4 of the second member 260, but because the thermal conductivity of air is much lower than that of metal, the third region 203, which has a thick second member 260 formed from the material (H) with a high thermal conductivity, can have a higher thermal conductivity than the second region 202, which has a thin second member 260.

Alternatively, as shown in FIG. 13, the second mold 200 may be configured so that the second member 260 is incorporated into the first member 250, which has a cylindrical shape, parallel to the longitudinal axis direction x4. In the case where multiple grooves 210 are provided in the circumferential direction z4, it is preferable that the second member 260 is also arranged in accordance with the position of the grooves 210 in the circumferential direction z4. In this case, in the second region 202, there is the first member 250 outside the radial direction y4 of the second member 260, but because the thermal conductivity of the first member 250 is lower than that of the second member 260, the third region 203, which has a thick second member 260 formed from the material (H) with a high thermal conductivity, can have a higher thermal conductivity than the second region 202, which has a thin second member 260.

The average value of the thickness of the third region 203 in the radial direction y4 of the second member 260 is preferably 1.2 times or more the average value of the thickness of the second region 202 in the radial direction y 4 of the second member 260. The average value of the thickness of the third region 203 in the radial direction y4 of the second member 260 is more preferably 1.5 times or more the average value of the thickness of the second region 202 in the radial direction y4 of the second member 260, even more preferably 2 times or more, particularly preferably 3 times or more, and preferably 10 times or less, more preferably 9 times or less, and even more preferably 8 times or less. Thus, by changing the thickness in the radial direction y4 of the second member 260 in the second region 202 and the third region 203, it is possible to easily adjust the magnitude of the thermal conductivity of the third region 203 relative to the thermal conductivity of the second region 202. This makes it possible to easily adjust the surface roughness of the base region 28b and tip region 28t of the formed balloon 2.

The material (L) with a low thermal conductivity constituting the first member 250 is preferably copper, aluminum, or an alloy thereof, and from the viewpoint of high thermal conductivity and strength, an alloy of copper and beryllium (beryllium copper) is more preferred. The material (H) that constitutes the second member 260 and has a higher thermal conductivity than the material (L) is preferably iron or an alloy thereof, and from the viewpoint of durability and strength, stainless steel is more preferred.

The present application claims priority based on Japanese Patent Application No. 2022-123511 filed on Aug. 2, 2022. All the contents described in Japanese Patent Application No. 2022-123511 filed on Aug. 2, 2022 are incorporated herein by reference.

### DESCRIPTION OF REFERENCE SIGNS

1: balloon catheter
2: balloon for balloon catheter
5: hub
6: fluid inlet
20: balloon body
21: proximal sleeve part
22: proximal tapered part
23: straight tubular part
24: distal tapered part
25: distal sleeve part
28: protrusion part
28b: base region
28B: base end
28t: tip region
28T: outer end
30: shaft
31: distal shaft
32: proximal shaft
50: guidewire port
60: inner shaft
70: tip member
80: radiopaque marker
100: first mold
100C: straight tubular part of the first mold
100S: sleeve part of the first mold
100T: tapered part of the first mold
105: inner cavity of the first mold
110: groove part of the first mold
110b: groove base region of the first mold
110t: groove tip region of the first mold
120: cylindrical wall part of the first mold
130: cathode conductive member
140: power source
141: cathode of the power source
142: anode of the power source
150: electrolytic solution
160: electrolytic bath
200: second mold
201: first region
202: second region
203: third region
205: inner cavity of the second mold
210: groove part of the second mold
210b: groove base region of the second mold
210t: groove tip region of the second mold
220: cylindrical wall part of the second mold
250: first member
260: second member
300: parison
301: first end of the parison
302: second end of the parison
305: lumen of the parison
308: protrusion part of the parison
O1: figure center of the inner cavity of the first mold
O2: figure center of the inner cavity of the second mold
x1: longitudinal axis direction of the balloon
y1: radial direction of the balloon
z1: circumferential direction of the balloon
x2: longitudinal direction of the parison
y2: radial direction of the parison
z2: circumferential direction of the parison
x3: longitudinal axis direction of the first mold
y3: radial direction of the first mold
z3: circumferential direction of the first mold
x4: longitudinal axis direction of the second mold
y4: radial direction of the second mold
z4: circumferential direction of the second mold

1. A balloon for a balloon catheter having a longitudinal axis direction and a radial direction, comprising:
a balloon body having an outer surface and an inner surface; and
a protrusion part that projects outward from the outer surface of the balloon body in the radial direction and extends in the longitudinal axis direction, wherein
the protrusion part comprises a tip region including an outer end in the radial direction and a base region located inward from the tip region in the radial direction in a cross-section perpendicular to the longitudinal axis direction;
the balloon body and the protrusion part are composed of the same material; and
a surface roughness of the base region is smaller than a surface roughness of the tip region where a surface roughness of the protrusion part is measured in a direction extending from the outer end of the tip region toward the balloon body through a lower end of the base region and perpendicular to the longitudinal axis direction, the surface roughness of the base region is a surface roughness of a portion having a reference length extending from the lower end of the base region toward the outer end of the tip region, and the surface roughness of the tip region is a surface roughness of a portion having a reference length extending from the outer end of the tip region toward the lower end of the base region.

2. The balloon for a balloon catheter according to claim 1, wherein a surface roughness of the balloon body is larger than the surface roughness of the base region of the protrusion part where the surface roughness of the protrusion part and the surface roughness of the balloon body are measured in the direction extending from the outer end of the tip region to the balloon body through the lower end of the base region and perpendicular to the longitudinal axis direction, and the surface roughness of the balloon body is a surface roughness of a portion having a reference length circumferentially extending from the lower end of the base region.

3. The balloon for a balloon catheter according to claim 1, wherein a surface roughness of the balloon body is larger than the surface roughness of the tip region of the protrusion part where the surface roughness of the protrusion part and the surface roughness of the balloon body are measured in the direction extending from the outer end of the tip region to the balloon body through the lower end of the base region and perpendicular to the longitudinal axis direction, and the surface roughness of the balloon body is a surface roughness of a portion having a reference length circumferentially extending from the lower end of the base region.

4. A balloon catheter comprising the balloon for a balloon catheter according to claim 1.

5. A method for producing the balloon catheter according to claim 4, comprising:
preparing a parison having a tubular shape and composed of resin;
preparing a mold having a longitudinal axis direction and a radial direction, and having an inner cavity extending in the longitudinal axis direction through which the parison is to be inserted, the inner cavity comprising a groove part recessed outwardly in the radial direction and extending in the longitudinal axis direction and a cylindrical wall part other than the groove part;
inserting a cathode conductive member having an outer shape along the cylindrical wall part in a cross-section in the longitudinal axis direction into the inner cavity of the mold to bring the cathode conductive member into contact with the cylindrical wall part without contacting the groove part;
connecting the cathode conductive member to a cathode of a power source and connecting an outer wall of the mold to an anode of the power source;
introducing an electrolytic solution into the inner cavity of the mold, outside the cathode conductive member in the radial direction;
supplying power from the power source to the cathode conductive member and the mold to electrochemically polish the inner cavity of the mold;
removing the electrolytic solution and removing the cathode conductive member from the inner cavity of the mold, followed by inserting the parison into the inner cavity of the mold; and
introducing a fluid into a lumen of the parison to inflate the parison and bringing an outer wall of the parison into contact with the groove part and the cylindrical wall part of the mold, wherein
in a cross-section perpendicular to the longitudinal axis direction, the groove part has a groove tip region with a longer distance from a figure center of the inner cavity of the mold in the radial direction and a groove base region with a shorter distance from the figure center in the radial direction than that of the groove tip region.

6. The method according to claim 5, wherein no gap is formed between the cathode conductive member and the cylindrical wall part when the cathode conductive member is brought into contact with the cylindrical wall part.

7. A method for producing the balloon catheter according to claim 4, comprising:
preparing a parison having a tubular shape and composed of resin;
preparing a mold having a longitudinal axis direction and a radial direction, and having an inner cavity extending in the longitudinal axis direction through which the parison is to be inserted, the inner cavity comprising a groove part recessed outwardly in the radial direction and extending in the longitudinal axis direction and a cylindrical wall part other than the groove part;
inserting the parison into the inner cavity of the mold;
heating the mold; and
introducing a fluid into a lumen of the parison to inflate the parison and bringing an outer wall of the parison into contact with the groove part and the cylindrical wall part of the mold, wherein
in a cross-section perpendicular to the longitudinal axis direction, the groove part has a groove tip region with a longer distance from a figure center of the inner cavity of the mold in the radial direction and a groove base region with a shorter distance from the figure center in the radial direction than that of the groove tip region;
in a cross-section perpendicular to the longitudinal axis direction, the mold has a first region with a low thermal conductivity, a second region with a higher thermal conductivity than the first region, and a third region with a higher thermal conductivity than the second region; and
in a cross-section perpendicular to the longitudinal axis direction, the first region is located at the cylindrical wall part, the second region is located at the groove tip region, and the third region is located at the groove base region.

8. The method according to claim 7, wherein the first region is composed of a first member formed from a material (L) with a low thermal conductivity, the second region and the third region include a second member formed from a material (H) with a higher thermal conductivity than the material (L), and a thickness of the second material of the third region in the radial direction is greater than a thickness of the second material of the second region in the radial direction.

9. The method according to claim 8, wherein the average thickness of the second member of the third region in the radial direction is 1.2 times or greater the average thickness of the second member of the second region in the radial direction.

10. The balloon for a balloon catheter according to claim 1, wherein each of the reference length for the surface roughness of the base region and the reference length for the surface roughness of the tip region is 100 μm.
